(19) United States
(12) Patent Application Publication
(10) Pub. No.: US 2007/0224302 A1
(43) Pub. Date: Sep. 27, 2007

(54) Maintaining Anabolic Hormone Profile During Weight Loss and Intense Exercise

(76) Inventor: Shawn M. Talbot, Draper, UT (US)

Correspondence Address: HOLME ROBERTS & OWEN, LLP, 299 SOUTH MAIN, SUITE 1800, SALT LAKE CITY, UT, 84111, US

(21) Appl. No.: 11/690,722

(22) Filed: Mar. 23, 2007

Related U.S. Application Data
(60) Provisional application No. 60/785,149, filed on Mar. 23, 2006.

(51) Int. Cl. A61K 36/18

(52) U.S. Cl. 424/773

(57) ABSTRACT

Eurycoma longifolia is used for reducing cortisol levels and raising testosterone levels in persons under stress. A method for promoting weight reduction includes administering to a human having a caloric deficit an effective dose of Eurycoma longifolia for reducing cortisol levels and raising testosterone levels relative to respective cortisol and testosterone levels that would result in the absence of the administration of the Eurycoma longifolia. A method for promoting improved endurance aerobic performance in a human includes administering, within thirty minutes before aerobic exercise or during the aerobic exercise, to a human subject an effective dose of Eurycoma longifolia for reducing cortisol levels and raising testosterone levels in the human subject relative to respective cortisol and testosterone levels that would result in the absence of the administration of the Eurycoma longifolia.

### CROSS-REFERENCE TO RELATED APPLICATIONS

This application claims the benefit of Provisional Application No. 60/785,149, filed Mar. 23, 2006, entitled “Maintaining Anabolic Hormone Profile During Weight Loss And Exercise Training,” which is incorporated by reference herein.

### BACKGROUND OF THE INVENTION

1. The Field of the Invention

The present invention relates to the fields of food compositions and dietary supplements. More particularly, the invention provides compositions and methods for promoting weight reduction in a human subject by administering an effective dose of Eurycoma longifolia to a human subject undergoing weight loss or exercise.

2. The Relevant Technology

One way of categorizing metabolic processes, whether at the cellular, organ or organism level is as anabolic or catabolic. Catabolism is the part of metabolism that breaks down molecules into smaller units to generate energy and simultaneously takes measures to conserve energy. Stress such as intense exercise and weight loss can put the body into the catabolic state, in which the body can experience muscle loss, reduced metabolic rate with a corresponding reduced calorie expenditure, and weight gain in the form of fat. One aspect of the catabolic state is that it can be characterized as having high cortisol (a catabolic stress hormone) levels and low testosterone (an anabolic hormone) levels. High cortisol levels have also been tied to psychological effects leading to higher caloric consumption, increased appetite, and negative moods. Conversely, in the anabolic state the body experiences muscle maintenance or growth, normal metabolic rates, and weight loss in the form of fat. The anabolic state can be characterized by relatively low cortisol levels and high testosterone levels.

As noted above, weight loss and intense exercise both place the body in a catabolic state with reducing testosterone levels and elevating cortisol levels. While these bodily responses serve their intended role for survival, they inhibit the ability of humans to lose weight or perform at a high level during lengthy strenuous exercise.

### BRIEF SUMMARY OF THE INVENTION

The present invention provides methods and compositions using an effective dose of Eurycoma longifolia for reducing cortisol levels and raising testosterone levels in a mammal, preferably a human, under physical stress. Reducing cortisol levels and raising testosterone levels in a human under weight loss induced physical stress reduces stress, keeps the body in an anabolic state, and can reduce appetite. Similarly, reducing cortisol levels and raising testosterone levels in a human under aerobic exercise induced physical stress, for example in endurance based athletics such as cycling and long distance running, helps keep the body in an anabolic state and improve continued aerobic performance.

One example embodiment of the invention is a method for promoting weight reduction in a human subject, the method generally including administering to a human an effective dose of Eurycoma longifolia for reducing cortisol levels and raising testosterone levels in a human subject in need thereof. The Eurycoma longifolia can be administered to the human subject as part of a regular regime of dose administrations where the timing and amount of each dosage is determined with respect to the cumulative effects of the regime. The effective dose can be defined in several ways depending on the application, for example as the amount sufficient to reduce cortisol levels by at least 5% and raise testosterone levels by at least 5% in a human undergoing weight loss relative to those levels without the supplement.

Another example embodiment of the invention is a method for promoting improved endurance aerobic performance in a human subject, the method generally including administering an effective dose of Eurycoma longifolia to a human subject within thirty minutes before aerobic exercise or during the aerobic exercise. The effective dose can be defined as the amount sufficient for creating a measurable improvement in exercise performance by a human user engaged in endurance aerobic exercise or alternatively as the amount sufficient to reduce cortisol levels by at least 5% and raise testosterone levels by at least 5% in a human undergoing strenuous aerobic exercise relative to those levels without the supplement.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

In the following description, numerous specific details are set forth in order to provide a thorough understanding of the present invention. It will be obvious, however, to one skilled in the art that the present invention may be practiced without these specific details. In other instances, well-known aspects of dietary supplements and topical creams have not been described in particular detail in order to avoid unnecessarily obscuring the present invention.

The present invention relates to the discovery and use of Eurycoma (Eurycoma longifolia, also known as Tongkat ali and Malaysian ginseng) for maintaining normal levels of cortisol and testosterone during weight loss and intense exercise training. More particularly, Eurycoma is used to help maintain normal (low) cortisol and normal (high) testosterone levels during the stress of weight loss and intense exercise.

Eurycoma longifolia is a traditional Malaysian herbal remedy used for increasing energy levels and libido in older men. According to the invention, effective compositions and methods of using Eurycoma have now been identified for the maintenance of an anabolic hormone profile during weight loss and intense exercise training. Individuals attempting to lose weight or promote their intense exercise performance, by way of example only intense endurance exercise such as long distance running, cycling, etc., may be treated with an effective dose of Eurycoma to enhance weight loss and promote exercise performance.

Accordingly, one embodiment of the invention is a composition of matter having an effective dose of Eurycoma longifolia for promoting weight loss in a mammal. For weight loss, effective doses of Eurycoma help prevent the body from seeking to gain weight by storing fat and increasing appetite. The Eurycoma as used according to the invention can thereby help stop the “yo-yo” diet effect where a dieter's initial weight loss of a few pounds sends the body into a catabolic state, leading to binge eating and fat storage. In particular, it is believed that inhibiting or reducing the cortisol spike associated with weight loss can make it easier for a human to control their appetite. Similarly, it is believed that controlling the cortisol and testosterone levels helps prevent the body from increasing fat storage.

Accordingly, an embodiment of the invention is a method of promoting weight loss in a mammalian subject undergoing weight loss that includes administering a composition of matter having from about 1 mg to about 1,000 mg of a Eurycoma root extract. A preferred Eurycoma root extract has from about 18% to about 28% eurypeptides. In a more preferred embodiment the composition of matter has from about 20 mg to about 200 mg of a Eurycoma root extract, preferably containing 18-28% eurypeptides. In a still more preferred embodiment the composition of matter has at least about 50 mg of the root extract, preferably containing 18-28% eurypeptides.

Of course, the foregoing and other Eurycoma amounts disclosed herein may increase or decrease depending on the concentration of the Eurycoma root as defined by the percent eurypeptides and the extraction technique. For example, different extraction techniques will likely result in different extraction products. Although not each embodiment of the invention is tied to any particular extraction process, a presently preferred extraction process is a hot water extraction process, which yields from 18% to about 28% eurypeptides confirmed by high-performance liquid chromatography (HPLC) analysis.

For weight reduction, a regular dosage regime is preferably, though not necessarily, implemented. The generation of appropriate dosage regimes and the timing of single use applications can be determined by one of skilled in the art in view of the disclosure herein. By way of example only, however, a human in a weight loss regime can preferably take one daily supplement within the foregoing ranges of Eurycoma. Alternatively, a human can take a single supplement.

Where the composition of matter is administered as part of a regime wherein the composition of matter is administered a plurality of times, the effective dose is preferably sufficient to create a cumulative relative weight loss by a human user of at least 1 pound, wherein a relative weight loss is defined by a human obtaining a lower weight than the human user would have obtained without receiving the effective dosage of Eurycoma longifolia.

Another embodiment of the invention is a composition of matter having an effective dose of Eurycoma longifolia for creating a measurable improvement in exercise performance by a human user engaged in endurance aerobic exercise. For exercise, the Eurycoma helps keep a human's testosterone levels high and cortisol levels low well into an exercise session and past the point in which a body usually shifts towards a catabolic state where testosterone levels decrease. As a result, a human's performance can continue at a higher level for a greater duration of their intense exercise session than it would without the Eurycoma.

Accordingly, a method of promoting improved exercise performance in a mammal engaged in intense exercise includes administering a composition of matter having from about 1 mg to about 1,000 mg of a Eurycoma root extract, preferably containing 18-28% eurypeptides. In a more preferred embodiment the composition of matter has from about 20 mg to about 200 mg of a Eurycoma root extract, preferably containing 18-28% eurypeptides. In a still more preferred embodiment the composition of matter has at least about 50 mg of the root extract, preferably containing 18-28% eurypeptides.

Where improved exercise performance is desired, either a regular dosage regime or a single dosage close to or during intense exercise can be used. The generation of appropriate dosage regimes and the timing of single use applications can be determined by one of skilled in the art in view of the disclosure herein. By way of example only, however, a human in a weight loss regime can preferably take one daily supplement within the foregoing ranges of Eurycoma within about 30 minutes of exercise. Alternatively, a human can take a dosage of Eurycoma during exercise if the exercise long enough for the body to process and receive the benefits of the Eurycoma. In addition, a human can take a daily or regular dosage as part of an extended regime to improve training potential and increase overall conditioning.

As used herein the term “an effective dose” indicates an amount sufficient to create a desirable result. For example, in one embodiment an effective dose is the amount necessary to obtain a measurable change in cortisol levels and testosterone levels. For example, an effective dose may therefore preferably reduce cortisol level by at least 5 percent and raise testosterone levels by at least 5 percent in a human engaged in weight loss or intense exercise, each value calculated relative to a change in cortisol or testosterone that would happen in the absence of taking the effective dose. For example, use of the herein disclosed supplements may reduce a cortisol increase from 10% to 0% during endurance exercise. Alternatively, an effective dose can be the amount necessary to suppress appetite in a human undergoing weight loss, promote a relative weight reduction, or improved exercise performance. The precise parameters of an effective dose may of course vary with a number of factors, including by way of example only weight, gender, age, and desired result.

A variety of suitable forms of providing for usage of the disclosed compositions can be used and provided. General examples include nutritional supplements, pharmaceutical preparations, foods supplemented with the specified compositions of the invention, injections, and topical treatments such as suitably formed creams, ointments, and lotions. Such packaging and administration forms for the compositions are well known and are not discussed in detail herein to avoid obscuring the invention.

Such administration forms may include, by way of example only: tablets, capsules, injections, topical creams, ointments, lotions, dietary supplement in either solid or liquid form, and a food additive. For example, for use in improving exercise performance Eurycoma can be added to a sports drink, gel, or exercise bar. As another example, for weight loss Eurycoma can be added to a diet shake.

Other conventional additives such as stabilizers, pH adjusters, excipients, carriers and diluents and the like may be added depending on the dosage form as is known in the art, provided they do not interfere with the activity of Eurycoma.

In addition, Eurycoma can be preferably combined with a variety of other supplements, vitamins, stabilizers, and the like as are known in the art and in view of the disclosure herein to create a desirable supplement or administration form, provided they do not interfere with the activity of Eurycoma. By way of example only, such additives can include other forms of ginseng, caffeine, guarana, green tea extract, chromium, vanadium, CLA, and the like.

The compositions of the invention can be formulated so as to provide quick, sustained or delayed release of the Eurycoma after administration employing procedures and formulations known in the art.

The following examples are shown by way of illustration only.

### EXAMPLES

### Example 1

A high endurance test was performed to identify the extent to which Eurycoma assisted in maintaining test subjects' normal anabolic state. A twenty-four hour mountain bike relay was organized with healthy subjects where each cyclist rode four two hour laps on an identical course. The subjects were divided into a fifteen member test group and a fifteen member control group. The subjects in the test group received four doses of a 100 mg of a root extract containing 22% eurypeptides, each dose taken 30 minutes before each of the four laps. The subjects in the control group received placebos. Salivary samples were taken from each subject before and after each lap, the cortisol levels in each group were measured, and the cortisol levels for each group were averaged. It was found that the test subjects that consumed the Eurycoma averaged 31% lower salivary cortisol exposure than the control subjects and 19% higher testosterone than the control subjects.

### Example 2

A single dose of a weight loss shake incorporating an effective dose of Eurycoma longifolia can be prepared by mixing a powder with the following components in water.

| Ingredient | Amount | |
|---|---|---|
| Eurycoma longifolia | 50 | mg |
| (22% eurypeptides) | | |
| Whey protein | 20 | grams |
| concentrate | | |
| Maltodextrin | 20 | grams |
| Fructose | 5 | grams |
| Lecithin | 2 | grams |
| Total | 47.05 | grams |

### Example 3

A single dose of an exercise drink incorporating an effective dose of Eurycoma longifolia can be prepared by mixing a powder with the following components in water.

| Ingredient | Amount | |
|---|---|---|
| Eurycoma longifolia | 100 | mg |
| (22% eurypeptides) | | |
| Maltodextrin | 10 | grams |
| Sucrose | 7 | grams |
| Dextrose | 7 | grams |
| Glutamine | 1,000 | mg |
| Leucine | 400 | mg |
| Isoleucine | 200 | mg |
| Valine | 400 | mg |
| Vitamin C | 120 | mg |
| Calcium | 100 | mg |
| Magnesium | 150 | mg |
| Chloride | 380 | mg |
| Sodium | 250 | mg |
| Potassium | 160 | mg |
| Total | 26.26 | grams |

### Example 4

An single dose of an energy supplement incorporating an effective dose of Eurycoma longifolia can be prepared as follows:

| Ingredient | Amount | |
|---|---|---|
| Eurycoma longifolia | 50 | mg |
| (22% eurypeptides) | | |
| Cordyceps sinensis | 1,000 | mg |
| Rhodiola rosea | 300 | mg |
| Eleutherococcus | 100 | mg |
| senticoccus | | |
| Withania somnifera | 100 | mg |
| Total | 1550 | mg |

### Example 4

A single dose of a weight loss supplement incorporating an effective dose of Eurycoma longifolia can be prepared as follows:

| Ingredient | Amount | |
|---|---|---|
| Eurycoma longifolia | 50 | mg |
| (22% eurypeptides) | | |
| Citrus flavonoids | 80 | mg |
| Green tea | 100 | mg |
| Black tea | 100 | mg |
| Oolong tea | 100 | mg |
| Scutellaria baicalensis | 250 | mg |
| Chromium | 100 | mcg |
| Vanadium | 10 | mcg |
| 5-HTP | 100 | mg |
| Total | 780.11 | mg |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for promoting weight reduction in a human subject, the method comprising administering to a human subject having a caloric deficit an effective dose of Eurycoma longifolia for reducing cortisol levels and raising testosterone levels in the human subject relative to respective cortisol and testosterone levels that would result in the absence of the administration of the Eurycoma longifolia.

2. The method of claim 1, wherein the Eurycoma longifolia is obtained by a hot water extraction process yielding 18-28% eurypeptides.

3. The method of claim 1, wherein the effective dose is defined as sufficient to reduce cortisol levels by at least 5% and raise testosterone levels by at least 5% in a human having a caloric deficit relative to respective cortisol and testosterone levels that would result in the absence of the administration of the Eurycoma longifolia.

4. The method of claim 1, wherein the effective dose comprises from about 1 mg to about 1000 mg of a Eurycoma longifolia root extract containing from about 18% to about 28% eurypeptides.

5. The method of claim 1, wherein the effective dose comprises from about 20 mg to about 200 mg of a Eurycoma longifolia root extract containing from about 18% to about 28% eurypeptides.

6. The method of claim 1, wherein the Eurycoma longifolia is administered to the human subject as part of a regular regime of dose administrations and the timing and amount of each dosage is determined with respect the cumulative effects of the regime.

7. The method of claim 6, wherein the effective dose is sufficient to create a cumulative relative weight loss by a human user of at least 1 pound over a first duration of the regime, wherein a relative weight loss is defined by a human obtaining a lower weight than the human user would have obtained without receiving the effective dosage of Eurycoma longifolia.

8. The method of claim 1, wherein the Eurycoma longifolia is in an oral form comprising a liquid shake or a powdered mix having one or more additional ingredients suitable for forming a shake when mixed with a liquid.

9. The method of claim 1, wherein the Eurycoma longifolia is in an oral form selected from the group consisting of a beverage, a tablet, a capsule, a powder, a confectionary, and a supplemented food.

10. The method of claim 1, wherein the Eurycoma longifolia is blended with a topical cream for topical administration and administering the effective dose of Eurycoma longifolia comprises topically administering the Eurycoma longifolia.

11. A method for promoting improved endurance aerobic performance in a human subject, the method comprising administering, within thirty minutes before aerobic exercise or during the aerobic exercise, to a human subject an effective dose of Eurycoma longifolia for reducing cortisol levels and raising testosterone levels in the human subject relative to respective cortisol and testosterone levels that would result in the absence of the administration of the Eurycoma longifolia.

12. The supplement of claim 11, wherein the Eurycoma longifolia is obtained by a hot water extraction process yielding 18-28% eurypeptides.

13. The method of claim 11, wherein an effective dose of Eurycoma longifolia is defined as the amount sufficient for creating a measurable improvement in exercise performance by a human user engaged in endurance aerobic exercise.

14. The method of claim 11, wherein the effective dose of Eurycoma longifolia is administered to the human subject in a beverage or food having additives selected to enhance endurance performance.

15. The method of claim 11, wherein the effective dose is defined as sufficient to reduce cortisol levels by at least 5% and raise testosterone levels by at least 5% in a human undergoing endurance aerobic exercise relative to respective cortisol and testosterone levels that would result in the absence of the administration of the Eurycoma longifolia.

16. The method of claim 11, wherein the effective dose comprises from about 2 mg to about 200 mg of a Eurycoma longifolia root extract containing from about 18% to about 28% eurypeptides.

17. The method of claim 11, wherein the Eurycoma longifolia is blended with a topical cream for topical administration and administering the effective dose of Eurycoma longifolia comprises topically administering the Eurycoma longifolia.

18. The method of claim 11, wherein the Eurycoma longifolia is in an oral form selected from the group consisting of a beverage, a tablet, a capsule, a powder, a confectionary, and a supplemented food.

19. The method of claim 11, wherein the Eurycoma longifolia is blended with a topical cream for topical administration and administering the effective dose of Eurycoma longifolia comprises topically administering the Eurycoma longifolia.
